(19) United States
(12) Patent Application Publication
(10) Pub. No.: US 2009/0030430 A1
(43) Pub. Date: Jan. 29, 2009

(54) Hair Plucking Apparatus

(76) Inventor: Osnat Levi, Jerusalem (IL)

Correspondence Address: The Law Office of Michael E. Kondoudis, PC, 888 16th Street, N.W., Suite 800, Washington, DC, 20006, US

(21) Appl. No.: 12/178,699

(22) Filed: Jul. 24, 2008

Related U.S. Application Data
(60) Provisional application No. 60/935,083, filed on Jul. 25, 2007.

(51) Int. Cl. A61B 17/50

(52) U.S. Cl. 606/133; 606/211

(57) ABSTRACT

An apparatus for plucking out hairs of a subject using a thread knots method. The apparatus may comprise at least one pair of pincer-parts rotatably connected through connecting means; and at least one thread. The pair of rotatably connected pincer-parts creates a structure that is capable of opening and closing when rotated by a user around said rotation mechanism, where the thread is held by the pincer-parts in such a manner that the thread is wrapped around itself creating at least one knot having intersection points. Upon rotating the pincer-parts around the rotation mechanism back and forth in opposite rotation directions, the knots respectively move along a movement-path, where the knots enable capturing the hairs of the subject by tightening around the subject's hairs and plucking them by moving along the movement path, when tightened.

### CROSS REFERENCE TO RELATED APPLICATION

This application claims the benefit of U.S. Provisional Patent Application 60/935,083, titled “Facial Hair Removal System”, filed Jul. 25, 2007, which is incorporated herein by reference in its entirety.

### FIELD OF THE INVENTION

The present invention relates generally to the field of hair removing apparatuses and more particularly to apparatuses for plucking out body hairs.

### BACKGROUND OF THE INVENTION

Removing of hair from various parts of the body is an ancient custom, carried out by women as well as men throughout the history and in various religions and cultures all over the world. Hair can be removed from the facial area (e.g. eyebrows hair or hairs that grow anywhere else on a subject's face); from the axilla area; and from the arms, legs and groins areas, for instance.

There are various methods for removing of hairs from various areas in a subject's body. Perhaps one of the most common methods of removing unwanted hair is to use a razor or other type of blade that shaves off the unwanted hair at skin level. Among the drawbacks of this method are that it is quite easy to cut oneself, the results are often rather short-lived and may require at least daily shaves, and this is not a practical method for precisely removing individual hairs.

Another method involves applying harsh chemicals to the area of the skin that has unwanted hair. The chemicals remain on the skin until the hair is dissolved or burned off. Use of this depilation method may cause chemical burns or allergic reactions or both. Furthermore, the results may be inconsistent or short-lived, requiring additional or frequent re-applications. Moreover, this method may be too harsh and inexact for removing facial hair.

Removing hairs from their follicles is another option. Areas where the hair was removed from the roots remains hairless longer than if other depilatory methods have been used. Tweezers allow the user to remove each hair individually, but is a time-consuming and imprecise endeavor when removing all of the hairs from a particular area. Waxing and sugaring involve the application of a wax or sugar solution to the skin that, when removed, removes the hair with it. While these methods are suitable for removing facial hair, they are also painful and the top layer of skin may be stripped along with the hair.

Threading is another method of removing hair from the follicles, most frequently used for removing facial hair. This method dates back to ancient Egyptian times and is quite common throughout Asia and India.

Threading requires a trained practitioner to hold a twisted intersection point of cotton thread in her teeth and hands. The practitioner then manipulates the twisted section of the intersection point in order to catch and remove the unwanted hairs. There are many benefits to threading. This precise, delicate, and gentle method of removing facial hair is less painful than waxing and sugaring, there are no chemicals or special tools required, and the practitioner has a great deal of control over exactly which hairs are to be removed.

However, threading is an art that requires a trained practitioner, and is difficult or even impossible to practice on oneself, and therefore, one must schedule time and spend money for the service. Furthermore, the art of threading is held by few skilled practitioners and in many countries, where plucking out hair is popular, there are hardly any people who can practice this art and skill.

### SUMMARY OF THE INVENTION

The present invention, in some embodiments thereof, provides an apparatus for plucking out hairs of a subject using a threading method for the removal of the subject's hair.

According to some embodiments of the invention, the apparatus may comprise at least one pair of pincer-parts rotatably connected through connecting means comprising at least one rotation-mechanism; and at least one thread, where the thread may be held by the pincer-parts in such a manner that the thread is wrapped around itself creating at least one knot having intersection points where parts of the thread intersect.

According to some embodiments of the invention, the pair of rotatably connected pincer-parts may be curved to create a curved structure that is capable of opening and closing like a pincer when rotated by a user around the rotation mechanism, where the knots of the apparatus are moveably held by the pincer-parts.

According to some embodiments of the invention, upon rotating of the pincer-parts around the rotation mechanism back and forth in opposite rotation directions, the knots may respectively move back and forth along a movement-path, defined by the configuration of the wrapped thread that is held by the pincer-parts, where the knots enable tightening and loosening alternatively while moving along the movement-path. The knots may enable capturing of a subject's hairs of the subject's body area (skin area) that is approximated adjacently to the movement-path of the knots, where the hairs are captured inside the each not and plucking of the hairs is carried out by the tightening of the knots around the captured hairs and pulling them with the moving of the knot along the movement-path.

According to some embodiments of the invention, the rotation mechanism may be a spring situated between each pair of pincer-parts enabling to move the apparatus from an open position in which the at least one knot is located at one end of the movement-path to a closed position in which the at least one knot is located at the other end of the movement-path, by moving the edges of the pair of pincer-parts, which serve as handles, closer to one another and apart from one another in an alternating manner.

According to some embodiments of the invention, a user may rotatable move the handles of the pincer-parts, while the user holds the apparatus against the subject's body area in which hairs are to be plucked, such that the movement-path of the knots is situated in that area.

According to some embodiments of the invention, the pincer-parts may comprise receiving-openings such as curved slits, or holes suited to receive and hold parts of the thread to allow the thread to be held tightly in the thread's configuration, creating tension in the thread. Each handle may comprise a receiving-opening enabling to receive parts of the thread and each of the other edges of the pincer-parts may comprise a receiving-opening suited to receive and hold the ends of the thread.

According to some embodiments of the invention, the receiving-openings of the part of the pincer-parts opposite to the handles, may be curved slits with a niche at their end, where each end of the thread comprises a stopper suited to be received and held by the niches.

According to some embodiments of the invention, the apparatus may further comprise an adjustable stretching means enabling to fasten to the thread and facilitate in farther stretching of the thread. The stretching means may enable adjusting the stretching level of the thread and thereby the tension force of the thread.

Additionally, the adjustable stretching means may be coiled spring with a hook connected to one end of the spring, where the other end of the spring may be connected to the connecting means. The spring may enable stretching to reach the thread and the hook may enable fastening the thread to the spring, to allow holding the thread in a stretched manner.

According to some embodiments of the invention, the outer side of the handles, which is used by the user to hold the apparatus and rotate the handles may comprise protrusions, to facilitate in increasing the roughness of the outer side of the handles to prevent the user's hands from slipping when holding said handles.

According to some embodiments of the invention, the rotation means may be operatively associated with a motor unit enabling to rotate the handles automatically, while the user holds the apparatus against a body area in which hairs are to be plucked, such that the movement-path of the knots is situated in that area.

According to some embodiments of the invention, the connecting means may further comprise a first locker and a second locker enabling to secure the rotation means in its position defining the pincer-parts' axis of rotation.

According to some embodiments of the invention, the second locker may be connected at the side of the rotation means, which is approximated to the subject body area where the first locker may be connected to the opposite side of the rotation means. The second locker may be positioned between the pincer-parts and may be sunken or hidden therein. The respective side of the pincer-parts may be closer to the subject's body area than the second lock once approximating the apparatus to the subject's body area.

According to some embodiments of the invention a multiplicity of pincer-parts pairs may be rotatably connected to the rotation means, each pair may hold a different thread, to facilitate in enlarging the subject's body area from which hairs are to be plucked.

According to some embodiments of the invention the frontal part of each pincer-part opposite to the part of the handle, may be arched, wherein the inner side of those frontal arched parts comprise cavities to facilitate the durability of the arched structure.

The present invention further provides a method for plucking of body hairs of a subject, using the hair plucking apparatus. According to this method, the user may be required to first position the apparatus and especially the movement-path of the knots of the thread against an area of the subject's body that the user wishes to pluck; and to rotatably move the edges of the pair of pincer-parts, which serve as handles, closer to one another and apart from one another in an alternating manner.

Additionally, the present invention provides a method enabling the user to attach the thread to the pincer-parts prior to using the apparatus. According to some embodiments of the invention the process may comprise placing each end of the thread in a receiving-opening situated at the frontal edge of each pincer-part (opposite the handles); wrapping the thread around itself to create the at least one knot; and stretching the thread and placing parts of the thread at the handles' receiving-openings.

Additionally, the user may fasten the thread part situated between the handles to the hook of the adjustment means to stretch the thread and adjust the thread's tension.

### BRIEF DESCRIPTIONS OF THE DRAWINGS

The subject matter regarded as the invention will become more clearly understood in light of the ensuing description of embodiments herein, given by way of example and for purposes of illustrative discussion of the present invention only, with reference to the accompanying drawings, wherein

FIG. 1 is a schematic illustration of a three-dimensional isometric front view of an apparatus for plucking of a subject's hairs, according to some embodiments of the invention;

FIG. 2 is a schematic illustration of a three-dimensional isometric back view of an apparatus for plucking of a subject's hairs, according to some embodiments of the invention;

FIG. 3A and FIG. 3B schematically illustrate side views of an apparatus for plucking of a subject's hairs, in a closed an open positions, respectively, according to some embodiments of the invention;

FIG. 4A, FIG. 4B and FIG. 4C schematically illustrate the process of placing a thread in the apparatus to allow the apparatus to hold the thread in a predefined configuration, according to some embodiments of the invention; and

FIG. 5 is a schematic illustration of an apparatus for plucking of a subject's hairs having a motor unit, according to some embodiments of the invention.

The drawings together with the description make apparent to those skilled in the art how the invention may be embodied in practice.

### DETAILED DESCRIPTIONS OF SOME EMBODIMENTS OF THE INVENTION

The present invention, in some embodiments thereof, provides an apparatus 100 for plucking out a subject's body hairs thereby removing them.

The apparatus 100 may be suited to pluck hairs of any area in the subject's body such as facial hair (e.g. eyebrows, mustache and the like), hairs in the groins and axilla (armpit) area and the like.

The subject may be any person which hairs are being plucked by the apparatus 100.

Any user, including the subject him/herself, may use the apparatus 100 to pluck the subject's hairs.

The apparatus 100 may be designed in various shapes and sizes to be suited for hair plucking of different areas of subjects' bodies. For example, an apparatus 100 for plucking facial hairs may be smaller than an apparatus 100 for axilla and groins hairs.

While the description below contains many specifications, these should not be construed as limitations on the scope of the invention, but rather as exemplifications of the preferred embodiments. Those skilled in the art will envision other possible variations that are within its scope. Accordingly, the scope of the invention should be determined not by the embodiment illustrated, but by the appended claims and their legal equivalents.

An embodiment is an example or implementation of the inventions. The various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments” or “other embodiments” means that a particular feature, structure, or characteristic described in connection with the embodiments is included in at least one embodiments, but not necessarily all embodiments, of the inventions. It is understood that the phraseology and terminology employed herein is not to be construed as limiting and are for descriptive purpose only.

The principles and uses of the teachings of the present invention may be better understood with reference to the accompanying description, figures and examples. It is to be understood that the details set forth herein do not construe a limitation to an application of the invention. Furthermore, it is to be understood that the invention can be carried out or practiced in various ways and that the invention can be implemented in embodiments other than the ones outlined in the description below.

It is to be understood that the terms “including”, “comprising”, “consisting” and grammatical variants thereof do not preclude the addition of one or more components, features, steps, or integers or groups thereof and that the terms are to be construed as specifying components, features, steps or integers. The phrase “consisting essentially of”, and grammatical variants thereof, when used herein is not to be construed as excluding additional components, steps, features, integers or groups thereof but rather that the additional features, integers, steps, components or groups thereof do not materially alter the basic and novel characteristics of the claimed composition, device or method.

If the specification or claims refer to “an additional” element, that does not preclude there being more than one of the additional element. It is to be understood that where the claims or specification refer to “a” or “an” element, such reference is not be construed that there is only one of that element. It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks. The term “method” refers to manners, means, techniques and procedures for accomplishing a given task including, but not limited to, those manners, means, techniques and procedures either known to, or readily developed from known manners, means, techniques and procedures by practitioners of the art to which the invention belongs. The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined. The present invention can be implemented in the testing or practice with methods and materials equivalent or similar to those described herein.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

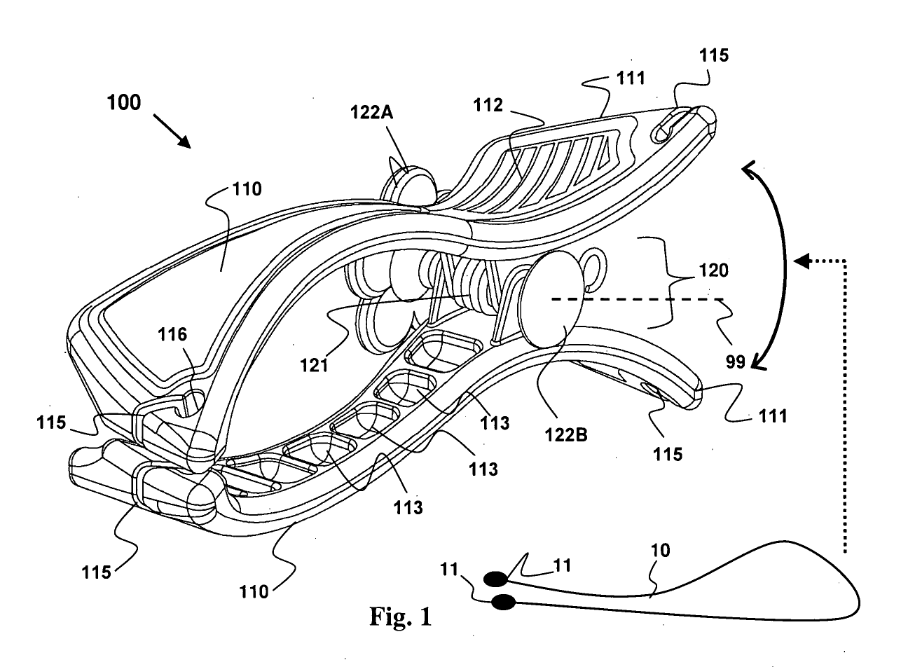

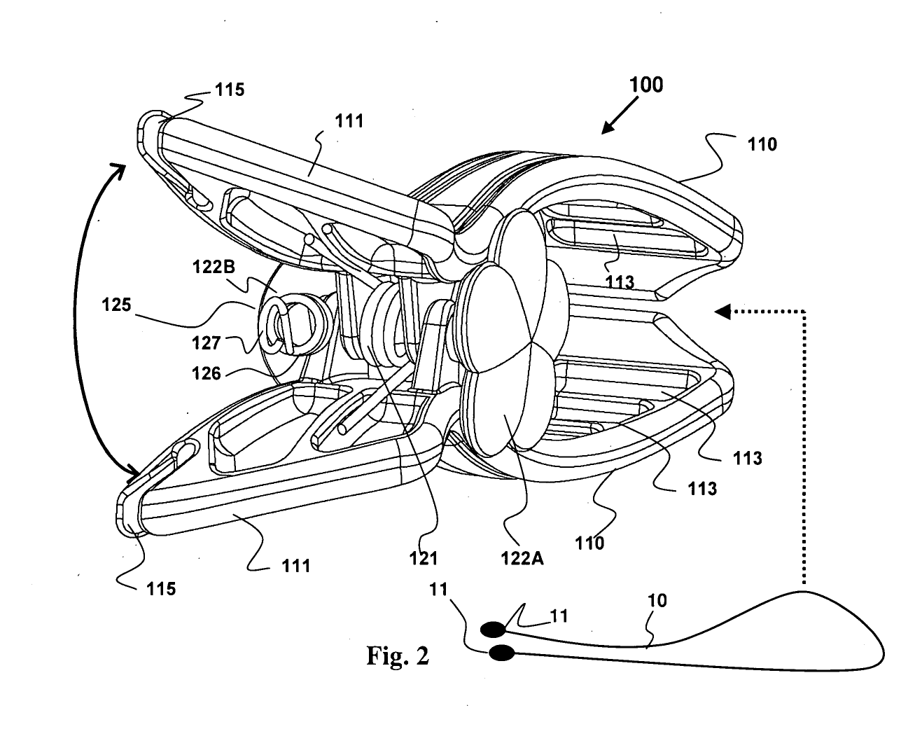

Reference is made to FIG. 1 and FIG. 2, which schematically illustrate a three-dimensional isometric front and back view, respectively, of the apparatus 100 for plucking of subject's hairs, according to some embodiments of the invention.

According to these embodiments, the apparatus 100 may comprise at least some of:
at least one pair of pincer-parts 110
connecting means 120 comprising at least one rotation-mechanism 121;
at least one thread 10; and
adjusting means 125.

One edge of each pincer-part 110 may serve as a handle 111, as illustrated in FIG. 1, enabling a user to hold these edges, using his/her fingers, to manually rotate the pincer parts 110 around a predefined axis of rotation 99, by moving the edges of the handles 111, closer to one another and apart from one another in an alternating manner. The other edges of the pincer-parts 110 may create a pincer-like space in which the thread can be held, where the plucking of the subject's hairs may be carried out by approximating one side of the apparatus 100 with the thread 10 held thereby, and coupling it to a desirable area of the subject's body (close to the skin of that area).

According to some embodiments of the invention, the handles 111 may be used to manually operate the apparatus 100 by alternatively pressing the handles 111 against each other (clutching the handles 111) and releasing the handles 111, allowing them to retract back to an open position. To press the handles 111, the user may be required to use the fingers of one of his/her hands for clutching of the handles 111.

According to some embodiments of the invention, as illustrated in FIG. 1 and FIG. 2, the pincer-parts 110 may be rotatably connected to one another through the connecting means 120. The pair of rotatably connected pincer-parts 110 may create a structure (e.g. a pincer-shaped structure) that is capable of opening and closing when rotated by a user around the rotation mechanism 121.

According to some embodiments of the invention, as illustrated in FIG. 1 and FIG. 2, the connecting means 120 may comprise a spring as the rotation means 121 having two edges each protruding and holding the inner surface of a pincer-part 110, where the spring may be rotatably mounted to a pivot inserted therethrough. The connecting means 120 may further comprise a first locker 122A and a second locker and 122B enabling to hold the pivot and spring 121 in place.

According to some embodiments of the invention, as illustrated in FIG. 1, the pincer-parts 110 may comprise receiving-openings 115 such as curved slits or holes, suited to receive and hold parts of the thread 10 to allow the thread 10 to be held tightly in the thread's 10 configuration. The handles 111 may comprise receiving-openings 115 slits that are suited to hold the middle part of the thread 10, where the receiving-openings 115 slits situated at the other edge of the pincer-parts 110 may be suited to receive the thread's 10 ends.

According to some embodiments of the invention, as illustrated in FIG. 1, the thread 10 ends may be connected to stoppers 11, which may be objects that are larger in diameter than the cross sectional diameter of the thread 10. The stoppers 11 may be made from any material known in the art such as plastic, rubber, the thread 10 materials and the like.

As illustrated in FIG. 1, the slits receiving-openings 115 of the frontal edges of the pincer-parts 110 may comprise a larger niche 116 to allow receiving the stoppers 11 and holding them therein.

The receiving-openings 115 may hold the thread 10 in a tight configuration to allow plucking of hairs, using thread tension.

According to some embodiments of the invention, as illustrated in FIG. 2, the adjustment means 125 may comprises a coiled spring 126 and a hook 127, where the hook 127 is connected to one end of the coiled spring 126 and the other end of the spring 126 is connected to the connecting means 120.

The adjustable stretching means 125 may fasten to the thread 10 and facilitate in farther stretching of the thread 10, once the thread 10 is already held by the receiving-openings 115.

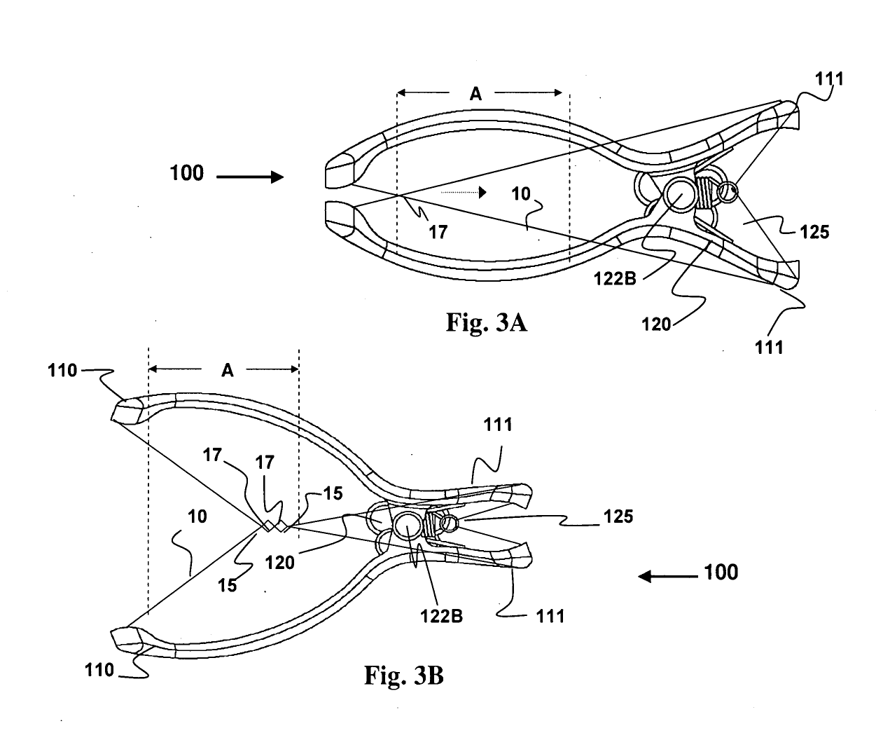

As illustrated in FIG. 3a and FIG. 3B, the coiled spring 126 may enable stretching to reach the thread 10 while the hook 127 enables fastening to the thread 10 and allow holding the thread 10 in a more stretched manner.

According to some embodiments of the invention, as illustrated in FIG. 1, the frontal part of each pincer-part 110 (opposite to the part of the handle 111) may be arched, where the inner side of those frontal arched parts may comprise cavities 113 to facilitate the durability of the structure, since the rotation of the handles 111 may apply pressure that can break those parts, depending on the size, structure and materials from which the pincer-parts 110 are made.

Additionally, or alternatively, as illustrated in FIG. 1, the outer parts of the handles 111 may be made from rough materials or comprise protrusions 112 to increase the roughness of the handles' 111 outer surface ad thereby to facilitate the user in holding and rotating the handles 111, since a smooth surface may cause the user's hand to slip from the handle 111.

According to some embodiments of the invention, as illustrated in FIG. 3A and FIG. 3B, the thread 10 may be held by the pincer-parts 110 in such a manner that the thread 10 is wrapped around itself creating at least one knot 17 having intersection points 15 where parts of the thread 10 intersect. The knots 17 may be moveably held by the pincer-parts 110 and the adjustment means 125.

The knot 17 may be a loop-shaped tie created by the wrapping of one part of the thread 10 on another.

When the apparatus's 100 pincer parts 110 are in a full closed position, as illustrated in FIG. 3A, the knots 17 of the thread 10 may reach a predefined first end spot, where the knots 17 may reach another second spot when reaching the full open position of the pincer-parts 110, as illustrated in FIG. 3B. The movement of the knot 17 from the open position to the closed position and vice versa defines a movement-path A. According to some embodiments of the invention, the alternatively changing in the positions (open and closed) may correspondently alternatively change the tension in each of the knots 17 since once side of the movement-path A may create less tension than the other thereby enabling the knots 17 to slightly open once shifting to one of the sides and then tightening back when shifting to the other side, due to the alternating opening and closing movement of the handles 111. The pincer-parts 110 may be curved, as illustrated in FIG. 1 and FIG. 2 to allow the thread to change the tension applied on the knot 17 when the knot 17 moves from one end of the movement-path A to the other.

The opening and closing of the pincer-parts 110 may be carried out by rotating of the handles 111 in opposite rotation direction—closer and apart from one another alternatively. Upon rotating of the pincer-parts 110 handles 111 around the rotation mechanism 121 back and forth in opposite rotation directions, the knots 17 in the thread 10 respectively moves back and forth along the movement-path A, where the knots 17 enables tightening while moving along the movement-path A, towards one of the movement-path's A ends and slightly loosening when moving towards the opposite ends of the movement-path A.

The knot 17 may enable capturing of the subject's hairs that are approximated to the movement-path A inside the knot 17 and plucking of the hairs by tightening around the captured hairs when reaching one end of the movement-path A and pulling them with the moving the knot 17 along the movement-path A towards the other end of the path A. By rotating the handles 111 back and forth with enough rotation speed and torque, the knots 17 of the thread 10 may tighten and loosen alternatively and thereby pluck whatever the knots 17 captures on their movement along movement-path A and then return to a more loosen position when returning back to one of the path's A ends and so forth.

As illustrated in FIG. 3A and FIG. 3B, the thread 10 may be held in such a manner that the knots 17 may slightly protrude one of the apparatus's 100 sides. This side is the one that should be approximated or coupled to the subject's skin and body area to perform the plucking process and hair removal.

According to some embodiments of the invention, as illustrated in FIG. 1, FIG. 3A and FIG. 3B, the second lock 122B may be connected at the side of the rotation means 121, which is approximated to the subject body area and the first locker 122A may be connected to the opposite side of the pivot 121. Therefore, the second locker 122B may be positioned between the pincer-parts 110 and sunken therein, to prevent the locker 122B from interfering the knots 17 from reaching the hairs of the subject when approximated to the subject's skin.

According to some embodiments of the invention, to pluck the subject's hairs from a predefined desirable body area, the user is required to first position the apparatus 100 and especially the protruding area of the knots 17 and movement-path A against the subject's selected skin area; and rotatably moving the edges of the pair of pincer-parts 110 (e.g. using the handles 111), closer to one another and apart from one another in an alternating manner.

The moving of the handles 111 enables the knots 17 to move back and forth along the movement-path A and thereby carry out the plucking of hairs, where subject's hairs that are positioned approximately to the movement-path A are captured inside the knot 17 and the moving of the knot 17 along the movement-path A enables tightening the knot 17 around the hairs and thereby plucking them by pulling the knot 17 further along the movement-path B.

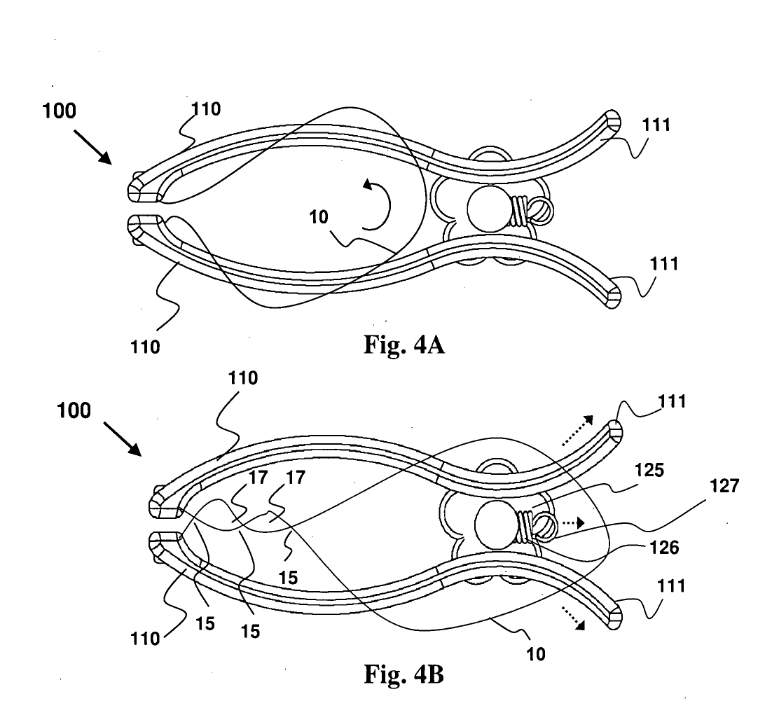

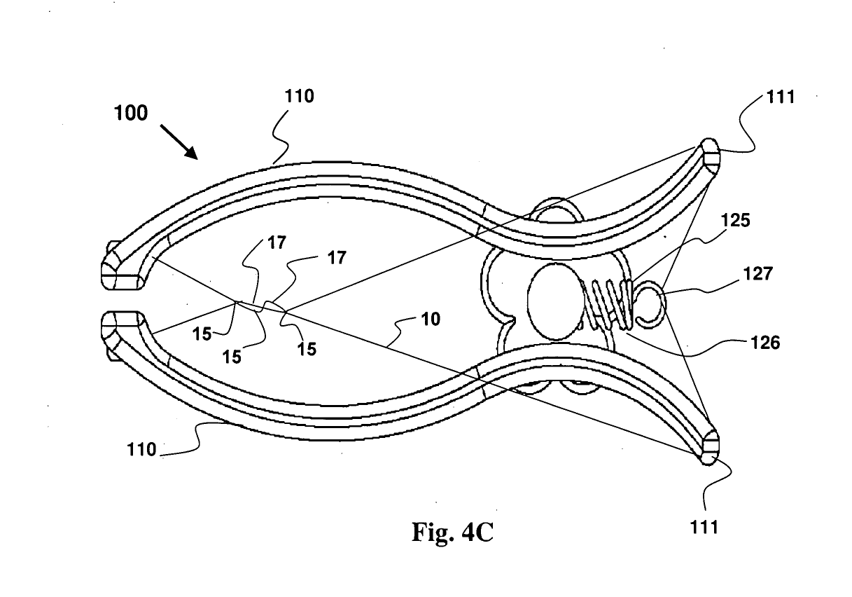

Reference is now made to FIG. 4a, FIG. 4B and FIG. 3C, which schematically illustrate a process for placing the thread 10 in the receiving-openings 115 and adjusting means 125. The process may comprise:
placing each end of the thread 10 (possibly by placing the stoppers 11 in the niches 116) in the receiving-opening 115 situated at the edge of each pincer-part 110, as illustrated in FIG. 4A;
wrapping the thread 10 around itself to create a at least one knot 17, as illustrated in FIG. 4B; and
stretching the thread 10 and placing parts of the thread 10 at the handles' 11 receiving-openings 115, as well as adjusting the stretching level and thereby the tension of the thread 10 that is held by the receiving-openings 115, as illustrated in FIG. 4C.

According to some embodiments of the invention, the adjustment may be carried out by stretching the coiled spring 126 towards the thread 10 part that is held between the handles 111 and fastening the thread 10 by the hook 127.

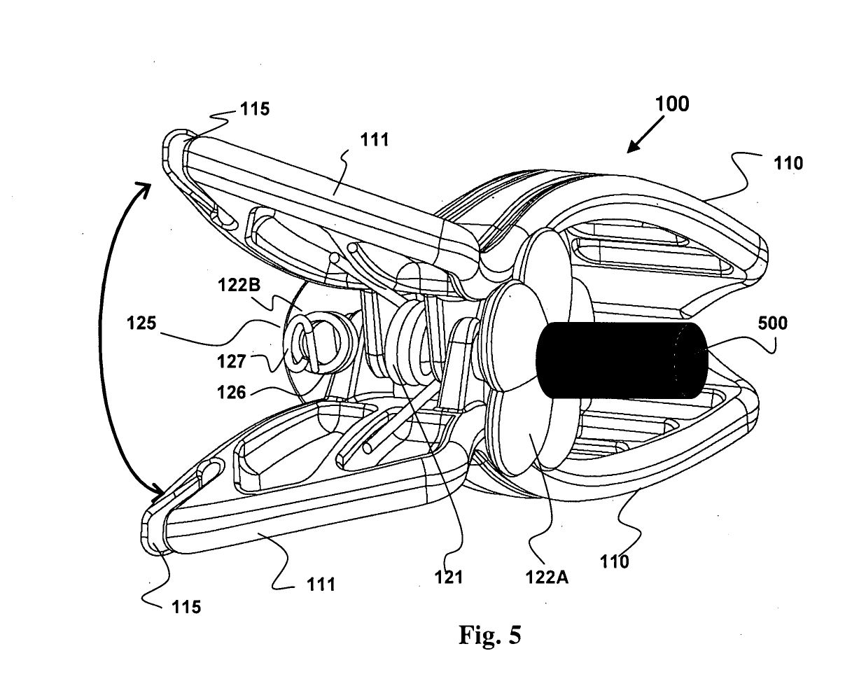

According to some embodiments of the invention, as illustrated in FIG. 5, the apparatus 100 may further comprise a motor unit 500, which may be operatively associated with the rotation mechanism 121 (e.g. the spring). The motor unit 500 may enable moving the pincer-parts 110 automatically, by automatically rotating the spring 121, while the user holds the apparatus 100 against a body area in which hairs are to be plucked, such that the movement-path A of the intersection point 15 is situated in that area. The motor unit 500 may comprise a motor, a shaft, rotated by the motor and a power source operating the motor, for example, enabling to apply torque to rotate the rotation means 121. The motor unit 500 may facilitate the user (which may be the subject) in operating the apparatus 100 applying less effort.

According to some embodiments of the invention, a multiplicity of pincer-parts 110 pairs may be rotatably connected to the rotation means 121, each pair may hold a different thread 10, to facilitate in enlarging the subject's body area from which hairs are to be plucked. The threads 10 may be of different length, type or thickness.

According to some embodiments of the invention, the pincer-parts 110 may be made from any solid material known in the art, such as plastic, wood, polymeric solids, metal, etc.

According to some embodiments of the invention, the thread 10 may be made from any materials known in the art that has high level of roughness, to allow high friction between the thread 10 and the subject's hairs, such as, for instance, cotton or any other fabric of low smoothness.

According to some embodiments of the invention, the thread 10 may be coated with and/or comprise soothing materials, enabling to sooth the skin of the subject while or after the knots 17 plunk the subject's hairs. The soothing materials may be any liquid, gel or solid materials known in the art that can sooth skin such as, for example, aloe vera gel coating the thread 10, chamomile powder or liquid absorbed in the thread 10, a mixture of soothing materials both absorbed and coating the thread 10 and the like.

While the invention has been described with respect to a limited number of embodiments, these should not be construed as limitations on the scope of the invention, but rather as exemplifications of some of the preferred embodiments. Those skilled in the art will envision other possible variations, modifications, and applications that are also within the scope of the invention. Accordingly, the scope of the invention should not be limited by what has thus far been described, but by the appended claims and their legal equivalents.

What is claimed is:

1. An apparatus 100 for plucking of hairs of a subject, said apparatus 100 comprising
at least one pair of pincer-parts 110 rotatably connected through connecting means 120 comprising at least one rotation-mechanism 121, wherein said pair of rotatably connected pincer-parts 110 creates a structure that is capable of opening and closing when rotated by a user around said rotation mechanism 121; and
at least one thread 10, wherein said thread 10 is held by the pincer-parts 110 in such a manner that the thread 10 is wrapped around itself creating at least one knot 17 having intersection points 15 where parts of the thread 10 intersect, wherein said at least one knot 17 is moveably held by the pincer-parts 110,
wherein, upon rotating of said pincer-parts 110 around said rotation mechanism 121 back and forth in opposite rotation directions, said at least one knot 17 respectively moves back and forth along a movement-path A, defined by the configuration of the thread 10 that is held by the pincer-parts 110, wherein said at least one knot 17 enables tightening while moving along said movement-path A,
wherein the knot 17 enables capturing of a subject's hairs that are approximated to said movement-path 10 inside said knot 17 and plucking of said hairs by tightening around said captured hairs and pulling them with the moving of said knot 17 along said movement-path A.

2. The apparatus 100 of claim 1, wherein rotation mechanism is a spring 121 situated between each pair of pincer-parts 110 enabling to move the apparatus 100 from an open position in which said at least one knot 17 is located at one end of the movement-path A to a closed position in which said at least one knot 17 is located at the other end of the movement-path A, by manually moving the edges of said pair of pincer-parts 110, which serve as handles 111, closer to one another and apart from one another in an alternating manner.

3. The apparatus 100 of claim 2, wherein the user moves said handles 111, while the user holds the apparatus 100 against a body area in which hairs are to be plucked, such that the movement-path A of the knot 17 is situated in that area.

4. The apparatus 100 of claim 3, wherein said pincer-parts 110 comprise receiving-openings 115 suited to receive and hold parts of the thread 10 to allow the thread 10 to be held tightly in the thread's 10 configuration,
wherein each said handle 111 comprises a receiving-opening 115 enabling to receive parts of the thread 10 and each of the other edges of the pincer-parts 110 comprises a receiving-opening 115 suited to receive and hold the end of the thread 10.

5. The apparatus 100 of claim 4, wherein said receiving-openings 115 are curved slits, wherein each end of said thread 10 comprises a stopper 11 suited to be received and held by at least two of said slits 115,
wherein the slits 115 at one edge of the pincer-parts 110 are suited to receive said stoppers 11 and the slits 115 at the other edge of the pincer-parts 110 are suited to receive and hold a part of said thread 10.

6. The apparatus 100 of claim 5 further comprising an adjustable stretching means 125 enabling to fasten to the thread 10 and facilitate in farther stretching of the thread 10, wherein said stretching means 125 enables adjusting the stretching level of the thread 10 and thereby the tension force of the thread 10.

7. The apparatus 100 of claim 6, wherein said adjustable stretching means 125 is a coiled spring 126 with a hook 127, wherein said hook 127 is connected to one end of said spring 126 and the other end of the spring 126 is connected to said connecting means 120,
wherein said spring 126 enables stretching to reach the thread 10 and said hook 127 enable fastening the thread 10 to said spring 126 to allow holding the thread 10 in a stretched manner.

8. The apparatus 100 of claim 2, wherein the outer side of said handles 111, which is used by the user to hold said apparatus 100 and rotate said handles 111, comprises protrusions 112 to facilitate in increasing the roughness of the outer side of the handles 111 to prevent the user's hands from slipping when holding said handles 111.

9. The apparatus 100 of claim 2, wherein said spring 121 is operatively associated with a motor unit 500 enabling to move said handles 111 automatically, while the user holds the apparatus 100 against a body area in which hairs are to be plucked, such that the movement-path A of the intersection point 15 is situated in that area.

10. The apparatus 100 of claim 1, wherein said connecting means 120 further comprise at least one first locker 122A enabling to secure the rotation means 121 in its position defining the pincer-parts' 110 axis of rotation.

11. The apparatus 100 of claim 10, wherein said thread 10 in its intersection pointed configuration is situated in such a manner that allows the user to approximate the at least one knot 17 to the subject's body area, from which hairs are to be plucked, as much as possible,
wherein said connecting means 120 further comprise a second locker 122B, wherein the second locker 122B is connected at the side of said rotation means 121, which is approximated to the subject body area and the first locker 122A is connected to the opposite side of said rotation means 121, and
wherein said second locker 122B is positioned between said pincer-parts 110 and is sunken therein, where the side of the pincer-parts 110 is closer to the subject's body area than said second lock 122B once approximating said apparatus 100 to the subject's body area.

12. The apparatus 100 of claim 1, wherein a multiplicity of pincer-parts 110 pairs are rotatably connected to said rotation means 121, each pair holds a thread 10, to facilitate in enlarging the subject's body area from which hairs are to be plucked.

13. The apparatus 100 of claim 1, wherein said pincer-parts 110 are made from solid materials, which is at least one of: plastic, wood, polymeric solids, or metal.

14. The apparatus 100 of claim 1, wherein said thread 10 is made from materials of high level of roughness, to allow high friction with the subject's hairs.

15. The apparatus 100 of claim 1, wherein said thread 10 is at least partly made from cotton.

16. The apparatus 100 of claim 1, wherein the frontal part of each pincer-part 110′ opposite to the part of the handle 111, is arched, wherein the inner side of those frontal arched parts comprise cavities 113 to facilitate the durability of the arched structure.

17. The apparatus 100 of claim 1 wherein said thread 10 comprises soothing materials enabling to sooth the skin of the subject while the knots 17 plunk the subject's hairs.

18. A method for plucking of body hairs of a subject, using a hair plucking apparatus 100 comprising at least one pair of pincer-parts 110 rotatably connected through connecting means 120; and at least one thread 10 wrapped around itself and held by said pincer-parts 110 in such a manner that creates at least one knot 17 comprising intersection points 15 held in tension by said pincer-parts 110, said method comprising:
positioning the apparatus 100 against an area of the subject's body; and
rotatably moving the edges of said pair of pincer-parts 110, which serve as handles 111, closer to one another and apart from one another in an alternating manner,
wherein said moving of the handles 111 enables the at least one knot 17 to move back and forth along a movement-path A, defined by the movement along which the at least one knots 17 of the thread 10 is moving, and
wherein subject's hairs that are positioned approximately to said movement-path A are captured inside said knot 17 and the moving the knot 17 along said movement-path A enables tightening the knot 17 around said hairs and thereby plucking them.

19. The method of claim 18, further comprising attaching the thread 10 to the pincer-parts 110 prior to using said apparatus 100, wherein said pincer-parts 110 comprise receiving-openings 115 suited to receive and hold parts of the thread 10 to allow the thread 10 to be held tightly in the thread's 10 configuration,
wherein each said handle 111 comprises a receiving-opening 115 enabling to receive and hold parts of the thread 10 and wherein each of the other edges of the pincer-parts 110 comprises a receiving-opening 115 suited to receive and hold the end of the thread 10, said process comprising the steps of:
placing each end of the thread 10 in a receiving-opening 115 situated at the other edge of each pincer-part 110;
wrapping the thread 10 around itself to create a at least one knot 17; and
stretching the thread 10 and placing parts of the thread 10 at the handles' 11 receiving-openings 115.

20. The method of claim 19 further comprises the step of adjusting the stretching level and thereby the tension of the thread 10 that is held by the receiving-openings 115, wherein said apparatus 100 further comprises stretching means 125 situated between said handles 111 and comprising a hook 127 and a coiled spring 126 which are connected, wherein said adjustment is carried out by stretching the coiled spring 126 towards the thread 10 part that is held between the handles 111 and fastening the thread 10 by said hook 127.

21. The method of claim 18, wherein a user manually and alternatively clutches said handles 111 to and from each other to rotate said pincer-parts 110 and thereby operate said apparatus 100.

22. The method of claim 18, wherein said apparatus 100 comprises a motor unit 500, which is operatively associated with said rotatable connecting means 120, wherein said motor unit 500 enables automatically rotating said pincer-parts 110.
